(19) United States
(12) Patent Application Publication
(10) Pub. No.: US 2026/0026678 A1
(43) Pub. Date: Jan. 29, 2026

(54) DISPOSABLE CONNECTOR FOR COUPLING A STEERABLE ENDOSCOPE TO A LARYNGOSCOPE

(71) Applicant: Covidien LP, Mansfield, MA (US)

(72) Inventors: Chenghao Bi, Boulder, CO (US); Michael Ng, Medindie (AU); Xin-An Chen, Broomfield, CO (US); Evan Tuer, Cupar (GB); Peter Inglis, Boulder, CO (US)

(73) Assignee: Covidien LP, Mansfield, MA (US)

(21) Appl. No.: 19/270,757

(22) Filed: Jul. 16, 2025

Related U.S. Application Data
(60) Provisional application No. 63/675,873, filed on Jul. 26, 2024.

(51) Int. Cl. A61B 1/00; A61B 1/005; A61B 1/267

(52) U.S. Cl. CPC A61B 1/00124; A61B 1/00006; A61B 1/0005; A61B 1/0057; A61B 1/267

(57) ABSTRACT

The technology relates to a medical system that includes a video laryngoscope, an endoscope connector removably attached to a housing of the laryngoscope, and a steerable endoscope having an electrical interface at the proximal end for receiving steering commands from the laryngoscope. The endoscope connector is attached to the video laryngoscope using one or more magnets and includes several additional mechanical features configured to inhibit lateral and vertical movement of the connector relative to the laryngoscope. The connector includes a port into which the proximal end of the endoscope is inserted. The connector includes an electrical interface that, when the connector is attached to the laryngoscope and the endoscope is inserted into the port of the connector, is in contact with both an electrical interface of the laryngoscope and an electrical interface of the endoscope, thereby providing an electrical connection between the laryngoscope and the endoscope.

### CROSS-REFERENCE TO RELATED APPLICATIONS

This application claims the benefit of U.S. Provisional Application No. 63/675,873 filed Jul. 26, 2024, entitled “Disposable connector for coupling a steerable endoscope to a laryngoscope,” which is incorporated herein by reference in its entirety. To the extent appropriate a claim of priority is made to the above disclosure.

### BACKGROUND

Laryngoscopes are commonly used to perform intubations on patients who require breathing assistance. During an intubation, the laryngoscope may be used to manipulate the anatomy of the larynx and structures associated with a patient's airway, in order to obtain a view sufficient for insertion of a breathing tube (e.g., an endotracheal tube) into the trachea. In some situations, the anatomy of the patient, or injury or other health condition of the patient, may prevent a clinician from obtaining a clear view of the larynx. In situations where intubation of a patient may be difficult, an endoscope may be used to aid visualization of the larynx and insertion of the breathing tube. An endoscope is a narrow, flexible tube that typically includes a light and camera at a distal end of the tube that is inserted into the body for visualizing anatomical structures of a patient. The combined use of a laryngoscope and endoscope may assist the clinician in performing an intubation.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter.

In an aspect, the technology relates to a medical video system that includes a video laryngoscope, a steerable endoscope, and an endoscope connector configured to mechanically and electrically connect the endoscope to the laryngoscope, as described below.

The laryngoscope includes a housing having a rear surface with an embedded first magnetic element, a protruding compartment having an electrical interface, and a recessed portion. The system further includes a steerable endoscope (e.g., a relatively long, thin tube) having a proximal end with motors for articulating a distal tip of the endoscope, and an electrical interface for electrical connection with the laryngoscope. The system further includes an endoscope connector configured to be removably attached to the rear surface of the housing of the laryngoscope, where the endoscope connector is configured to electrically couple the electrical interface of the endoscope with the electrical interface of the laryngoscope. The endoscope and endoscope connector may be disposable elements, while the laryngoscope may not be disposable.

The endoscope connector includes a port (e.g., a tubular portion having a hollow center) configured to receive and enclose at least a portion of the proximal end of the endoscope. The endoscope connector includes an electrical passthrough between a first electrical interface (located inside the port and configured to be connected to the electrical interface of the endoscope) and a second electrical interface (located outside the port and configured to be connected to the electrical interface of the laryngoscope). The first electrical interface and second electrical interface may be electrically connected via an electrical passthrough. The electrical interface of the endoscope is aligned with the first electrical interface of the endoscope connector when the endoscope is fully inserted into the port of the endoscope connector.

The endoscope connector includes a second magnetic element that is used to attach the endoscope connector to the housing of the laryngoscope by placing the second magnetic element in contact with the portion of the housing in which the first magnetic element is embedded. The endoscope connector includes a protruding portion (e.g., a ledge or ridge) that is configured to fit snugly into (e.g., reside within) the recessed portion of the laryngoscope housing when the first and second magnetic elements are aligned and coupled via magnetism (e.g., when the endoscope connector is attached to the housing), thereby inhibiting both lateral and vertical movement of the endoscope connector relative to the housing of the laryngoscope. The endoscope connector includes a cupped portion that is configured to partially enclose the protruding compartment of the housing when the first and second magnetic elements are aligned and coupled. The cupped portion has an opening at a top surface of the cupped portion through which the first electrical interface of the endoscope connector can be accessed. This opening aligns with the electrical interface of the laryngoscope, thereby allowing the first electrical interface of the endoscope connector to make contact with the electrical interface of the laryngoscope.

The endoscope connector includes a flange opposite a first edge of a body portion, where the flange is configured to be in contact with a first edge of the housing when the first and second magnetic elements are aligned and coupled. The flange inhibits lateral movement of the endoscope connector relative to the laryngoscope housing, thereby improving its stability.

The endoscope connector's mechanical features ensure easy and secure attachment to a rear surface of the laryngoscope housing. Such features may enable the clinician to attach the endoscope connector to the laryngoscope during an intubation procedure without unduly disturbing the laryngoscope (which could lead to patient injury) while helping to maintain the sterility of the laryngoscope by physically shielding the laryngoscope from the endoscope. For example, the magnetic element, protruding portion, flange, and cupped portion of the endoscope connector are effective for implementing a stable, easily achieved attachment-yet are inexpensive, making them particularly appropriate for a disposable device.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIGS. 1A-1B depict views of an example of a video laryngoscope.

FIG. 2 depicts an example of a steerable endoscope.

FIGS. 3A-3C depict views of an example of an endoscope connector.

FIG. 4 depicts another example of an endoscope connector.

FIG. 5A depicts an exploded view of a combination of a laryngoscope, endoscope connector, and steerable endoscope.

FIG. 5B depicts an example combination of a laryngoscope, endoscope connector, and steerable endoscope.

FIG. 6 depicts a block diagram of an example combination of a laryngoscope, endoscope connector, and steerable endoscope.

### DETAILED DESCRIPTION

A laryngoscope is commonly used during the intubation of a patient who may require breathing assistance. An intubation is a medical procedure in which a clinician inserts a breathing tube (e.g., an endotracheal tube) into the mouth of the patient, past the larynx, and into the trachea. The breathing tube may then be connected to a ventilator or other device for supplying breathing gases to the patient. A laryngoscope may be used during intubation to help the clinician manipulate portions of the patient's anatomy, such as the tongue and epiglottis, and obtain a view of the larynx sufficient for inserting the breathing tube into the trachea. To further help visualize the larynx, some laryngoscopes may be configured with a video camera. A laryngoscope that includes a video camera may be referred to as a video laryngoscope (VL). Using a VL to view the larynx or other structures may be referred to as indirect-view laryngoscopy.

With some patients, performing an intubation may be difficult due to a variety of factors, such as inability to position the head or neck of the patient (e.g., due to injury), airway obstruction, atypical anatomy of the patient, other health considerations, or a combination of these or other factors. In these types of scenarios, clinicians may augment the use of a VL with a steerable endoscope, which is a narrow flexible tube that typically includes its own video camera system integrated into a steerable distal tip that is inserted into the patient's body. The endoscope may be a bronchoscope that is configured for viewing a patient's lungs and air passages, for example.

The endoscope may be navigated into the airway and/or lungs and positioned such that it provides supplemental visualization of the airway and/or lungs, and/or facilitates insertion of the breathing tube. In some examples, the breathing tube is passed over the endoscope and into position in the airway, with the endoscope itself serving as a channel or guide for inserting the breathing tube.

Some steerable endoscopes include their own display screens as well as an external device for controlling the distal tip, such as a control stick or other type of directional controller. During intubation, it can be difficult for a single medical professional to view multiple screens (e.g., display screens for the laryngoscope and endoscope) and manipulate multiple devices (a video laryngoscope and endoscope) as well as potentially an endotracheal tube.

The present disclosure describes systems and methods for a medical video system that combines a video laryngoscope and a steerable endoscope, resulting in a single instrument that may simplify and improve clinical workflows such as intubation, airway visualization, and endoscopic procedures. Examples of this instrument include a steerable endoscope that operatively couples to a detachable, disposable endoscope connector, which serves as a mechanical interface between the steerable endoscope and a VL, and which is detachable from the VL. The VL also includes an electrical interface through which electrical power is provided, through the endoscope connector, to the endoscope and through which communication signals are transmitted between the VL and steerable endoscope. For instance, power provided by the VL is received by the steerable endoscope through the endoscope connector, and used to operate a camera, light source, sensors, and/or other electronic features of the endoscope. Video and/or sensor data from the steerable endoscope is transmitted to the VL through the endoscope connector.

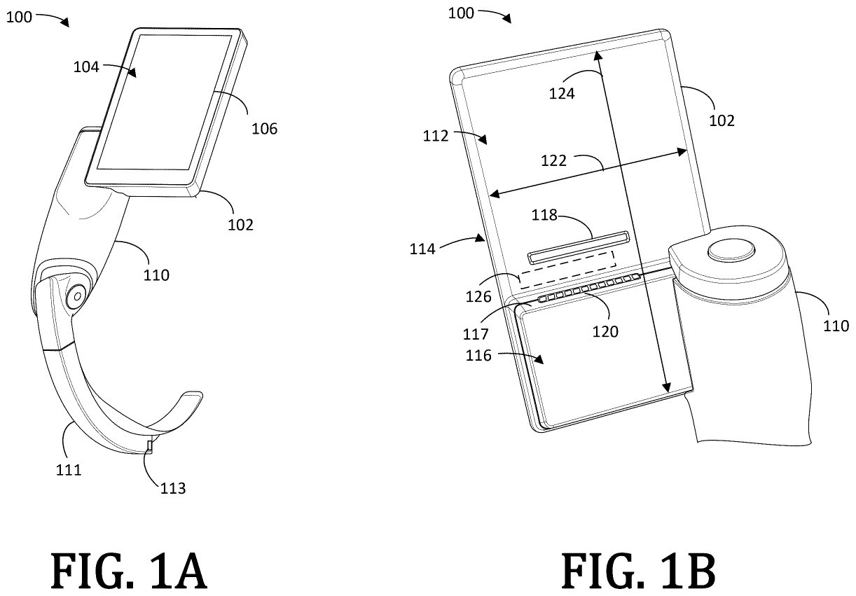

The detachable, disposable endoscope connector described herein may enable the clinician to attach the endoscope connector, also referred to as a shield or interposer, to the laryngoscope during an intubation procedure without unduly disturbing the laryngoscope handle (which could lead to patient injury) while helping to maintain the sterility of the laryngoscope handle and the endoscope. For example, the endoscope and endoscope connector may be disposable elements while the laryngoscope is reusable. The endoscope connector may help protect the laryngoscope housing from contact exposure to the endoscope. Additional details regarding a disposable endoscope connector for connecting a steerable endoscope to a laryngoscope are described with reference to FIGS. 1A-6.

FIGS. 1A-1B depict views of an example video laryngoscope (VL) 100. The VL 100 includes a housing 102 that includes a handle 110. The VL 100 also includes an arm or extension 111 that extends from the handle and includes a camera 113 positioned at the distal end of the arm or extension 111. The VL 100 may include additional functions or features typically associated with direct-view or indirect-view laryngoscopy, such as a power source (e.g., a battery), processor, and other electronic components.

The housing 102 has a front surface 104 and a rear surface 112. The front surface 104 includes and/or encloses a display 106 for displaying images for a clinician, such as images received from the camera 113 of the VL 100 and/or from a camera of an endoscope. Thus, the front of the VL 100 may be considered the direction that the display faces, and the rear of the VL100 may be considered the opposite direction. The display 106 may also be considered to define a plane. The direction perpendicular to that plane may be described frontward (e.g., outward from the front of the VL 100) or rearward (e.g., outward from the rear of the VL 100). The VL 100 may also be considered to have components that are proximal or distal from one another. While the VL 100 moves during use, the top of the display 106 (e.g., the portion of the display further from the blade) may be considered to be positioned proximal to the bottom of the display 106.

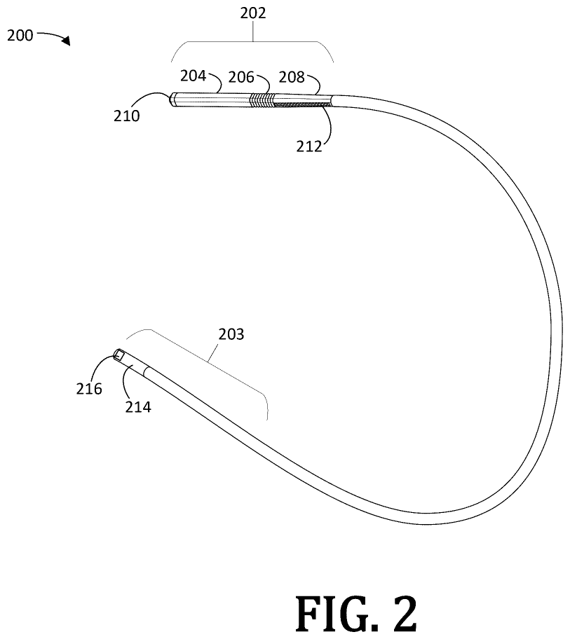

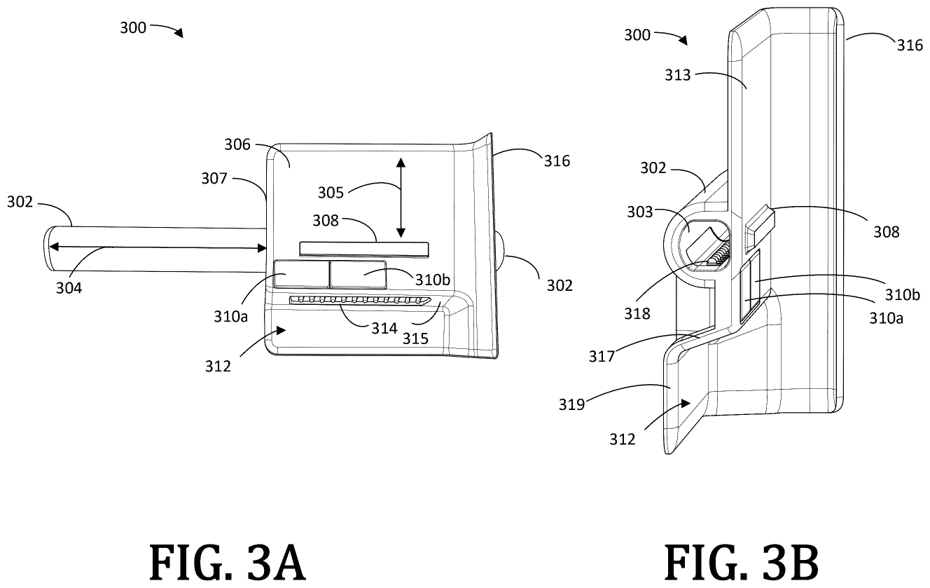

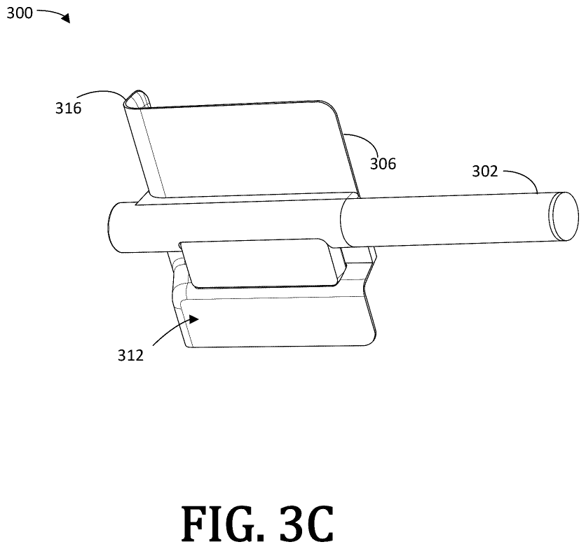

The rear surface 112 includes a protruding compartment 116 having a first electrical interface 120 for communicating with an endoscope via an endoscope connector (discussed with reference to FIGS. 2-3C). The protruding compartment 116 protrudes rearwardly from the planar portion of the rear surface 112. The protruding compartment 116 defines a shelf 117 on the proximal side compartment 116. The shelf 117 may define a surface that substantially perpendicular to planar portion of the rear surface 112. In some examples, the shelf 117 may be sloped at downward angle relative the planar portion of the rear surface 112. For instance, the large angle defined between a plane defined by the planar portion of the rear surface 112 and a plane defined by the shelf may be between 91-120 degrees. The first electrical interface 120 is positioned on the shelf 117. In the example depicted, the first electrical interface 120 includes a plurality of contact pads arranged in a row. Each contact pad may provide for the transfer of different signals or data. In some examples, the first electrical interface 120 includes at least five or 10 discrete contact points (e.g., pins or pads).

The rear surface 112 also includes a recessed portion 118 (or recess) for receiving a corresponding protruding portion of an endoscope connector. The recessed portion is recessed into the rear surface 112. The laryngoscope housing has a first edge 114, a horizontal width along a first axis 122 perpendicular to the first edge 114, and a vertical height along a second axis 124 perpendicular to the first axis. A magnetic element 126 is at least partially or entirely embedded within (e.g., enclosed by) the housing 102 of the VL 100. In some examples, the magnetic element is a permanent magnet or a magnetic material (such as a rare earth magnet). The magnetic element 126 may be positioned between the shelf 117 and the portion 118.

FIG. 2 depicts a steerable endoscope 200 having a proximal end 202 (e.g., an end of the laryngoscope that is closest to the video laryngoscope and the clinician when in use) and a distal end 203 (e.g., an end that is farthest from the laryngoscope and the clinician when in use). The proximal end 202 may include approximately 5, 15, 20, or 30 cm of the endoscope 200 as measured from a proximal tip 210 of the endoscope. The proximal end 202 includes a first rigid section 204 (e.g., starting at or near a proximal tip 210 of the endoscope), a second rigid section 208, and a flexible section 206 disposed between the first rigid section 204 and second rigid section 208. The first rigid section 204 may have a length of less than 2, 3, 5, or 10 cm. The second rigid section 208 may have a length of less than 2, 3, 5, or 10 cm. The flexible section may have a length of less than 2, 3, 5, or 10 cm. In some examples, the flexible section 206 may be smaller in length that the first rigid section 204 and/or the second rigid section 208. The flexible section 206 may allow the proximal end 202 of the endoscope 200 to be maneuvered through curved spaces, such as through an endotracheal tube, for example.

At the distal end 203 of the endoscope 200 is a steerable tip 214 which includes one or more accessories 216. The accessories 216 may include sensors such as an inertial measurement unit (IMU), which may provide measurement data associated with the acceleration, angular velocity, position, and/or other variables associated with position/orientation/movement of the steerable tip 214. The accessories 216 may further include lights, instrument ports, and/or a camera that captures endoscope image data.

The first rigid section 204 and second rigid section 208 may each include (e.g., enclose) one or more motors for steering the steerable tip 214 of the endoscope via pull wires, such as described in U.S. patent application Ser. No. 18/600,914, titled Disposable Endoscope Driver, which is incorporated herein by reference in its entirety. To the extent that application or reference numerals in that application conflict with the present application, this current application shall control.

The second rigid section 208 includes an electrical interface 212 on a surface of the endoscope 200 for receiving electrical power and/or steering commands from the VL 100. The electrical interface 212 may be electrically connected to the accessories 216 by a conductive element (not shown) enclosed within the endoscope 200. The electrical interface 212 provides a data path for transmitting sensor data, video images, and/or other types of data from the endoscope 200 to the VL 100. For instance, video data captured by a camera of the endoscope 200 may be transmitted to the VL 100 via the endoscope electrical interface 212 when the endoscope 200 is connected to the VL 100 by an endoscope connector (as described in more detail with reference to FIGS. 3A-6). In some examples, signals or data (such as clock, enable, timing, and/or other signals that are used to enable communication, configure accessories, and/or perform other operational functions) may be transmitted by the VL 100 through the electrical interface 212 in order to enable or configure the operation of the endoscope 200.

The electrical interface 212 may include a plurality of conductive pads, receptacles, pins, balls, ports, and/or other conductive elements used for establishing electrical connection to corresponding elements of the VL 100 (e.g., electrical interface 120) via an endoscope connector. The number of the discrete contacts of the endoscope electrical interface 212 may be the same number of discrete contacts of the electrical interface 120 of the laryngoscope 100. Alternatively or additionally, wireless communication components may be incorporated into the endoscope 200 to wirelessly communicate data between the endoscope 200 and the VL 100. In some examples, power to the motors within the endoscope may still be provided via electrical contacts even where wireless data transfer occurs. In still other examples, wireless power transfer may occur.

As mentioned above, the steerable tip 214 is controlled by motors at the proximal end 202 of the endoscope 200. The motors may be coupled with a first pair of pull wires that are used to move the steerable tip 214 in a first plane (e.g., left and right). The motors may also be coupled with a second pair of pull wires that are configured to move the steerable tip 214 in a second plane (e.g., up and down) that is substantially orthogonal to the first plane. The pull wires are routed along the length of the steerable endoscope 200 and are connected to opposite sides of the steerable tip 214. Accordingly, tensioning the different pull wires causes the steerable tip 214 to articulate in the corresponding direction.

The endoscope 200 may be connected to the VL 100 using an endoscope connector, which is described in detail with reference to FIGS. 3A-3C.

FIGS. 3A-3C depict views of a detachable endoscope connector 300 that can be used to mechanically and electrically couple an endoscope 200 to a VL 100 (e.g., without the endoscope being in contact with the VL 100 to maintain sterility of the VL 100). More specifically, FIG. 3A depicts a front view of the endoscope connector 300 showing the surfaces of the endoscope connector 300 that contact the rear surface 112 of the VL 100. FIG. 3B depicts a side view of the endoscope connector 300. FIG. 3C depicts a perspective view of the endoscope connector 300.

The endoscope connector 300 attaches to the rear surface 112 of the housing of the VL 100 and is configured to receive the proximal end 202 of the steerable endoscope 200 in a port 302.

In the example of FIGS. 3A-3C, the endoscope connector 300 includes two magnetic elements 310a, 310b for coupling with (e.g., magnetic attachment to) the magnetic element(s) 126 of the VL 100. In other examples, the endoscope connector 300 may include fewer or more magnetic elements. The two magnetic elements 310a, 310b may include permanent magnets or magnetic elements. The magnetic element(s) 310 may also be any magnetic (e.g., ferrous) material that is attracted to the permanent magnet 126 in the VL 100.

The endoscope connector 300 includes a body 306 and a port 302 configured to receive and at least partially enclose the proximal end 202 of the endoscope 200 when the endoscope 200 is inserted into an opening 303 of the port 302. For example, the port 302 may enclose at least a portion of the first rigid section 204, the flexible section 206, and/or the second rigid section 208 of the proximal end 202 of the endoscope 200. In the example depicted, the port 302 is tubular housing with an inner lumen that receives the proximal end 202 when inserted into the opening 303 of the port 302. The tubular housing of the port 303 may also protrude outwardly from an upper planar portion 313 of the body 306.

The cross section of the lumen of the port 302 and/or the opening 303 of the port 302 may not be symmetrical or entirely circular, as shown in FIG. 3B. For instance, the cross section(s) of the port 302 may match the outer geometry of the proximal end 202 of the steerable endoscope 200, which may also not have a symmetrical cross section. As a result, the proximal end 202 can only be inserted into the opening 303 in one orientation. That orientation results in the endoscope electrical interface 212 of the steerable endoscope 200 being aligned with an electrical interface 318 within the port 302. In other examples, the endoscope electrical interface 212 of the steerable endoscope 200 may be configured as ring contacts that wrap around the circumference of the steerable endoscope 200. In such examples, the proximal end 202 may be inserted in any orientation and the endoscope electrical interface 212 will contact the first electrical interface 318 of the endoscope connector 300.

The port 302 of the endoscope connector 300 includes the first electrical interface 318 that is connected via an electrical passthrough to a second electrical interface 314. The first and second electrical interfaces may each include a plurality of conductive pads, receptacles, pins, balls, ports, and/or other conductive elements. The second electrical interface 314 is accessible via an opening in a surface 315 of a cupped portion 312 of the body 306 of the endoscope connector 300. That is, the cupped portion 312 includes an opening along the surface 315 through which the second electrical interface 314 is accessible and can be electrically coupled with the electrical interface 120 of the VL 100. For example, the second electrical interface 314 connects to the electrical interface 120 on the VL 100 when the endoscope connector 300 is magnetically attached to the VL 100. The body 306 of the endoscope connector 300 may have a width that is less than the horizontal width of the housing of the video laryngoscope.

The first electrical interface 318 and second electrical interface 314 are conductively coupled via an electrical passthrough, which may be implemented as conductive pins or wires that extend from the first electrical interface 318 (located inside the port 302) to the second electrical interface 314 (located outside the port 302). In the example of FIGS. 3A-3C, the body 306 of the endoscope connector 300 encloses the electrical passthrough, thereby protecting the pins or wires from physical contact.

The body 306 of the endoscope connector 300 is connected along a longitudinal length of the port 302 (e.g., parallel to a longitudinal axis 304 of the port 302) and extends above and below the port along a vertical axis 305 that is perpendicular to the longitudinal axis 304 of the port 302. In some examples, the body 306 has a first width along the longitudinal axis 304 of the port that is narrower than a width of the port 302; for example, the port 302 extends beyond a first edge 307 of the body 306, where the first edge 307 is perpendicular to the longitudinal axis 304 of the port.

In some examples, the body 306 of the endoscope connector 300 includes a protruding portion 308 (such as a ridge or ledge) that is configured to fit snugly into the recessed portion 118 of the rear surface 112 of the VL 100 and inhibit lateral movement (e.g., horizontal, along the longitudinal axis 304 of the port) and vertical movement (e.g., along the vertical axis 305 that is orthogonal to the longitudinal axis 304 of the port) when the endoscope connector 300 is attached to the VL 100. The protruding portion 308 (or protrusion) protrudes in a direction towards the VL 100 (e.g., in a frontward direction). The protrusion may be positioned above the magnetic element(s) 310 and frontward of the port 302.

The endoscope connector 300 further includes a flange 316 opposite the first edge 307 of the body 306 to inhibit lateral movement when the endoscope connector 300 is attached to the VL 100. The portion forming the flange 316 may extend in a direction that is substantially perpendicular to the planar portion of the endoscope connector 300 that is above (e.g., proximal) to the port 302. The inner surface of the flange 316 is configured to contact a sidewall of the VL 100.

The cupped portion 312 protrudes rearwardly from an upper planar portion 313 of the endoscope connector 300. For instance, an extension segment 317 may extend rearwardly from the upper planar portion 313. A lower segment 319 then extends downward from the rearward-most point of the segment 317. Thus, the extension segment 317, the lower segment 319, and the lower portion of the flange 316 define a partial cavity that receives the protruding compartment 116 of the VL 100.

In some examples, the body 306 and/or port 302 of the endoscope connector 300 is/are fabricated from a plastic material (or other relatively non-conductive material) and may be translucent.

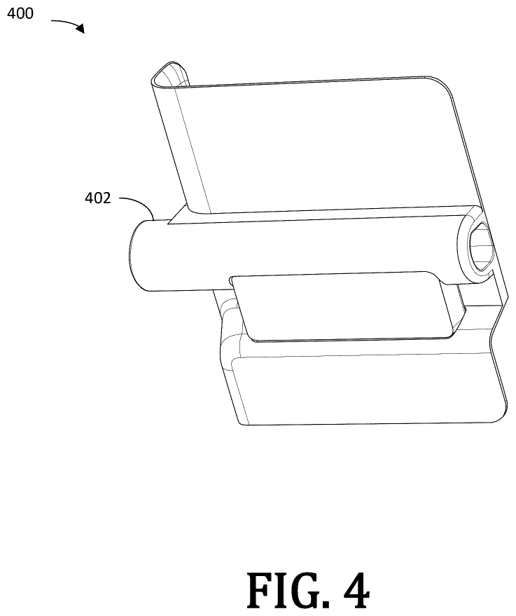

FIG. 4 is an example of an endoscope connector 400 having similar elements as shown in FIGS. 3A-3C but having a shorter port 402 relative to the port 302 depicted in FIGS. 3A-3C. For instance, the port 302 may have a longer tubular housing to encapsulate more of the proximal end 202 of the steerable endoscope 200. The port 302 may also have a closed end to further encapsulate the proximal end 202. In contract, the port 402 may have an open end on both sides. With the port 402, a portion of the proximal end 202 may extend beyond the tubular housing of the port 402 when the proximal end 202 of the steerable endoscope 200 is inserted into the port 402.

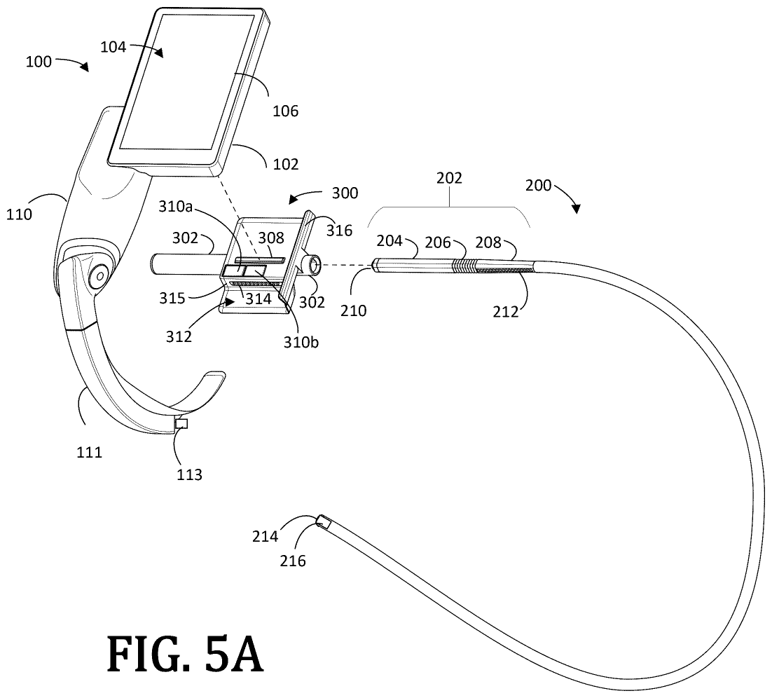

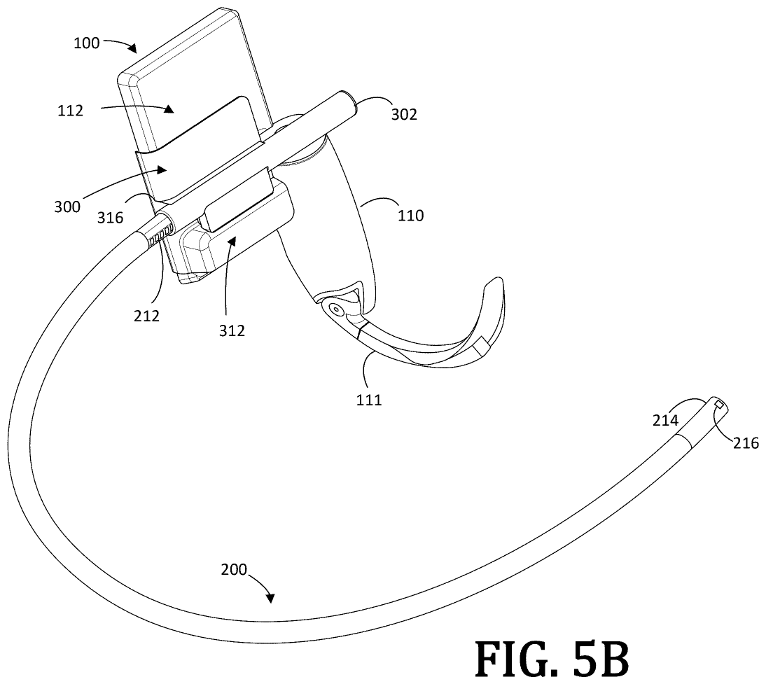

FIGS. 5A-5B depict views of a combined laryngoscope, endoscope, and endoscope connector. FIG. 5A depicts an exploded view of the laryngoscope 100, endoscope connector 300, and endoscope 200 roughly aligned for connection, while FIG. 5B depicts a combination of a laryngoscope 100, endoscope connector 300, and endoscope 200 when the endoscope connector 300 is attached to the laryngoscope 100 and the endoscope 200 is almost fully inserted into the endoscope connector 300.

As depicted in FIGS. 5A and 5B, the endoscope connector 300 may be coupled to the VL 100 by aligning the magnetic elements 310a, 310b of the endoscope connector 300 with the magnetic element(s) 126 in the housing of the VL 100. When the endoscope connector 300 is connected to (attached to) the VL 100, the protruding portion 308 of the endoscope connector 300 fits into (resides within) the recessed portion of the VL 100 and the flange 316 of the endoscope connector rests against a first edge 114 of the VL 100. The cupped portion 312 of the endoscope connector 300 fits over (e.g., partially encloses) the protruding compartment 116 of the VL 100 such that the second electrical interface 314 of the endoscope connector 300 is in contact with the electrical interface 120 of the VL 100. The port 302 of the endoscope connector 300 is parallel to the first axis 122 of the rear surface of the VL 100.

The proximal end 202 of the endoscope 200 may be inserted into the port 302. In one example, the endoscope 200 may slide into receiving elements of the port 302 that retain the endoscope 200 in the endoscope connector 300. In other examples, the endoscope 200 may be snap fit into the port 302 of the endoscope connector 300 or may be magnetically retained to port 302 of the endoscope connector 304 via retention magnets (not shown), among other approaches. The endoscope connector 300 may be designed to allow the endoscope 200 to be inserted into the endoscope port 302 at a depth that results in alignment of the electrical interface 212 of the endoscope 200 with the first electrical interface 318 of the endoscope connector 300. For instance, the port 302 may include a mechanical stop that blocks further insertion of the endoscope 200 when the electrical interface 212 of the endoscope 200 is aligned with the electrical interface 318 inside the port 302 of the endoscope connector 300. Another example is rails or protrusions that guide the endoscope into the port 302. When the proximal end 202 of the endoscope 200 is inserted into the port 302 to an extent that properly aligns corresponding electrical elements of the endoscope 200 and endoscope connector 300, the endoscope 200 may be considered to be in a fully inserted position. In examples, the endoscope connector 300 includes a feature that retains the proximal end 202 of the endoscope 200, when fully inserted into the endoscope port 302, such as with internal springs, clips, or other elements that apply force to portions of the proximal end 202 to retain it against a removal force.

As mentioned above, when the endoscope proximal end 202 is fully inserted into to the port 302 of the endoscope connector 300, the electrical interface 212 of the endoscope 200 is conductively connected to the corresponding first electrical interface 318 of the endoscope connector 300. When the endoscope connector 300 is, in turn, coupled to the VL 100 as described below, the second electrical interface 314 of the endoscope connector 300 is electrically coupled to the electrical interface 120 of the VL 100. In this manner, the endoscope 200 is electrically coupled to the VL 100 via electrical interface 120, electrical interface 314, an electrical passthrough, and electrical interface 318, and the VL 100 can control the endoscope steerable tip 214 through the endoscope connector 300.

In an example, the VL 100 receives data (such as video images) from the endoscope 200 via the endoscope connector 300 and displays the received data on the display 106. The display 106 may be capable of displaying images from multiple cameras simultaneously, such as images from the VL camera 113 and an endoscope camera (e.g., attachment 216), such as by split screen, picture-in-picture, or other display methods. The display 106 may be any of a variety of display technologies, such as LCD, LED, OLED, or other display technology. In examples, the display 106 may be a touch-sensitive display (e.g., a capacitive touch-sensitive display) that allows user input to be received through the display 106. Further, the display 106 may receive steering inputs from a user of the VL 100 for control of the endoscope steerable tip 214, or in some examples, the user may provide steering inputs via input keys or buttons associated with the VL 100. Elements of the VL 100 may translate the control inputs to corresponding motor outputs, which are transmitted to motors in the endoscope 200 through the endoscope connector 300 for control of the steerable tip 214.

The VL 100 may be capable of detecting the presence/absence of the endoscope connector 300 and/or steerable endoscope 200 (or vice versa). When detected, the VL 100 may energize power and signal connections to the endoscope connector 300 and/or steerable endoscope 200. In one example, the endoscope connector 300 and/or steerable endoscope 200 includes a magnetic element, and the VL 100 includes a sensor capable of detecting the magnetic element (such as a Hall effect sensor).

In operation, the steerable endoscope 200 is inserted into the port 302 of the endoscope connector 300. The endoscope 200 may be pre-loaded with a breathing tube, or the breathing tube may be post-loaded. During use, the clinician may control the endoscope steerable tip 214 by providing touch inputs via display 106. The VL 100 translates these steering inputs into steering commands that are provided to the endoscope 200 via electrical interfaces 120, 314, 318, 212. Accordingly, as compared to other systems, a single operator (e.g., clinician) may control both the video laryngoscope and endoscope without needing the assistance of another person to hold or guide the endoscope and/or a separate control system for the endoscope.

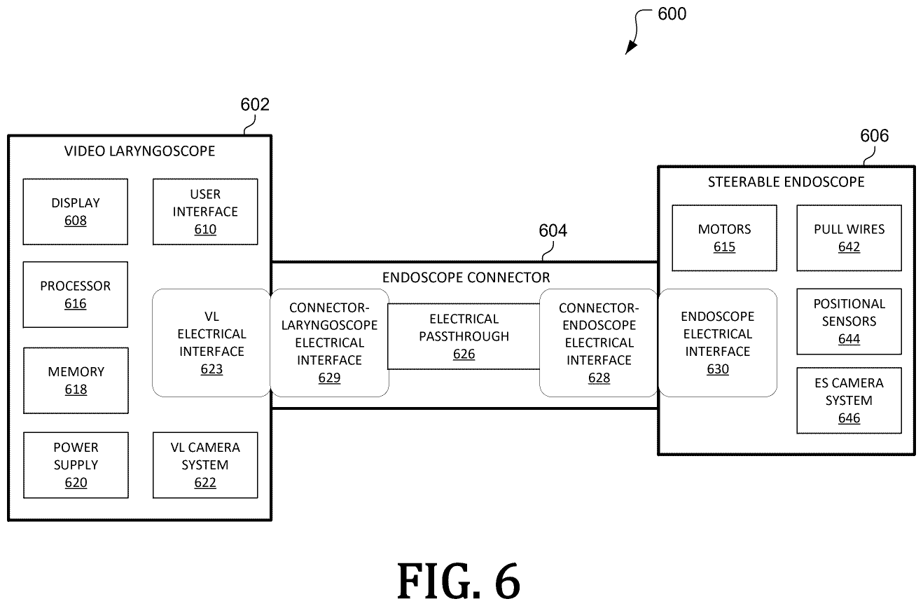

FIG. 6 is a block diagram of an example system 600 that includes a video laryngoscope 602, endoscope connector 604, and endoscope 606. Aspects of example system 600 may be similar to, or the same as, aspects of FIGS. 1A-5, above.

The proximal end of the endoscope 606 may include an electrical interface 630 for receiving electrical power and transmitting/receiving communication signals. The endoscope 606 includes one or more motors 615 for steering the distal tip of the endoscope 606. The motors 615 in the endoscope may be a type of DC motor, such as servo motors, stepper motors, or other type of motors, and may include analog and/or digital circuitry associated with providing electrical power and/or control of the motors 615. The motors 615 may include elements that couple to pull wires 642 or other elements used to control the steerable tip.

The VL 602 provides electrical power and motor control to the motors 615 via the VL electrical interface 623, which is electrically connected to endoscope electrical interface 630 through electrical interface elements of the endoscope connector 604. As depicted in example system 600, the endoscope connector 604 may include an electrical passthrough 626 that connects a first electrical interface 629 (which may be an example of electrical interface 314) with a second electrical interface 628 (which may be an example of electrical interface 318).

In example system 600, the user may provide steering control input through the display 608 (which may be a touch-sensitive display). Elements of the display 608, user interface 610, and/or processor 616 may translate the received steering control inputs to corresponding control signals for controlling the motors 615. These control signals may be transmitted to the motors 615 via the VL electrical interface 623 and the electrical interfaces of the endoscope connector 604 and steerable endoscope 606.

In examples, the endoscope connector-VL electrical interface 629, electrical passthrough 626, and/or endoscope connector-endoscope electrical interface 628 may include passive or active components such as those discussed herein. In some examples, the endoscope connector 604 may include additional electronic components (e.g., sensors or other components) that connect to the endoscope connector-VL electrical interface 629, electrical passthrough 626, and/or endoscope connector-endoscope electrical interface 628, and receive electrical power and/or transmit/receive signals to elements of the VL 602 and steerable endoscope 606.

The VL 602 also includes a processor 616, which may include one or more general purpose processors, microprocessors, microcontrollers, graphics processing unit (GPU), digital signal processors (DSPs), or other programmable circuits. In examples, the processor 616 may include any combination of commercially available components, and/or custom or semi-custom integrated circuits, such as application specific integrated circuits (ASICs). The processor 616 may include elements needed for control or communication with the display 608, user interface 610, motors 615, memory 618, power supply 620, VL camera system 622, and/or VL electrical interface 623. The processor 616 may perform control, interface, communication, or other processing functions by executing instructions that are stored in the memory 618. For instance, the memory 618 may store instructions that, when executed by the processor 616, cause the elements of the system 600 to perform operations described herein. The memory 618 may include RAM, ROM, electrically erasable programmable read-only memory (EEPROM), flash memory or other memory technology.

Further, the processor 616 and/or elements of the VL electrical interface 623 may direct or manage functions associated with electrical communication between the VL 602, endoscope connector 604, and/or steerable endoscope 606. In examples, the processor 616 may be associated with the processing and display of video images received from the ES camera system 646 through the VL electrical interface 623. The processor 616 may also be associated with the reception and processing of sensor data, such as data produced and received from motor/drum sensors 612, drum sensing subsystem 640, positional sensors 644, and/or other source of sensor data.

In some examples, the processor 616 and/or elements of the VL electrical interface 623 may manage or participate in a signal communications interface or protocol for transmitting and receiving data. For example, data may be transmitted/received between the VL 602, endoscope connector 604, and steerable endoscope 606 via serial peripheral interface (SPI), inter-integrated circuit (I2C), and/or other type of data transfer interface or protocol. Elements of the processor 616 and/or VL electrical interface 623 may provide or receive support signaling for data transmission/reception, such as clock(s), timing, enable, and/or other types of signals required for data transmission/reception.

Video images captured by the VL camera system 622 may be directed by the VL camera system 622 or by the processor 616 (or other element of VL 602) to be displayed on display 608. For instance, the display 608 may be capable of simultaneous display of video images from the VL camera system 622 and from endoscope camera 646, or the display 608 may be capable of switching between either source of video images.

The display 608 may further be used to display status, data, or other types of information associated with the operation of VL 602, endoscope connector 604, and/or steerable endoscope 606. For example, the display 608 may indicate presence/absence of the endoscope connector 604 and/or steerable endoscope 606. The display 608 may also indicate poor connection between the VL 602, endoscope connector 604, and/or steerable endoscope 606. In other examples, the display 608 may display sensor data (such as orientation of the endoscope steerable tip), motor output data, fault conditions, steering-related data, and/or any other indications associated with status, function, and/or operation of example system 600.

In examples where the display 608 provides for steering control of the endoscope steerable tip (such as when the display 608 is a touch-sensitive display), the user interface 610 may include a steering feature as part of the above-described GUI. Input steering control may be received through the GUI by the user interface 610 and translated by the user interface 610, processor 616, and/or elements of the motors 615 into corresponding control input to the motors 615.

The VL 602 includes a power supply 620, such as a battery, which may be housed in a suitable compartment of the VL. For example, a battery may be contained in the handle of the VL 602. The power supply 620 may further include analog or digital circuitry associated with control, regulation, and/or distribution of electrical power to elements of the VL 602, endoscope connector 604, and/or steerable endoscope 606.

Those skilled in the art will recognize that the methods and systems of the present disclosure may be implemented in many manners and as such are not to be limited by the foregoing aspects and examples. For instance, while the above examples are primarily discussed in relation to an endoscope, an introducer may be used, and an endoscope may be considered one example of an introducer in some examples. Further, any number of the features of the different aspects described herein may be combined into single or multiple aspects, and alternate aspects having fewer than or more than all of the features herein described are possible. Functionality may also be, in whole or in part, distributed among multiple components, in manners now known or to become known. Further, as used herein and in the claims, the phrase “at least one of element A, element B, or element C” is intended to convey any of: element A, element B, element C, elements A and B, elements A and C, elements B and C, and elements A, B, and C.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A medical video system comprising:
a video laryngoscope comprising a housing, a display, and a processor, wherein the housing comprises a rear surface comprising a protruding compartment, a first electrical interface on the protruding compartment, a recessed portion;
an endoscope connector configured to be removably attached to the rear surface of the housing, the endoscope connector comprising:
a port configured to at least partially enclose a proximal end of an endoscope, the port comprising a second electrical interface inside the port and a third electrical interface outside the port, wherein the second electrical interface and third electrical interface are conductively coupled by an electrical passthrough and the second electrical interface is in contact with the first electrical interface when the endoscope connector is attached to the rear surface of the housing; and
a body attached along at least a portion of a length of the port and extending above and below the port, the body comprising:
a flange opposite a first edge of the body, the flange configured to be in contact with a first edge of the housing of the video laryngoscope to inhibit lateral movement of the endoscope connector when the endoscope connector is attached to the rear surface, and
a protruding portion configured to reside within the recessed portion of the rear surface of the housing when the endoscope connector is attached to the rear surface.

2. The medical video system of claim 1, wherein the port is a tubular housing with an asymmetrical opening.

3. The medical video system of claim 1, wherein the port extends in a direction that is orthogonal to a plane formed by the flange.

4. The medical video system of claim 1, wherein the video laryngoscope further comprises a permanent magnet and the endoscope connector further comprises a magnetic material attracted to the permanent magnet, wherein the permanent magnet and the magnetic material are in proximity when the endoscope connector is connected to the video laryngoscope.

5. The medical video system of claim 1, wherein:
the endoscope connector includes a cupped portion configured to at least partially receive the protruding compartment of the video laryngoscope, wherein the cupped portion includes an opening through which the third electrical interface is in contact with the first electrical interface when the endoscope connector is attached to the video laryngoscope.

6. The medical video system of claim 1, wherein the housing encloses a rear surface of the display screen.

7. The medical video system of claim 1, further comprising a steerable endoscope having a proximal end that is configured to be removably installed in the port of the endoscope connector, wherein the proximal end comprises a fourth electrical interface configured to be in contact with the second electrical interface inside the port when the proximal end is installed in the port of the endoscope connector.

8. The medical video system of claim 7, wherein the proximal end comprises a first rigid portion extending from a tip of the proximal end, a second rigid portion, and a flexible portion disposed between the first rigid portion and the second rigid portion.

9. The medical video system of claim 8, wherein the second rigid portion comprises the fourth electrical interface and a motor configured to steer a distal tip of the endoscope via a pull wire connected to the motor and the distal tip.

10. The medical video system of claim 1, wherein the processor is programmed to receive first image data from a camera of the endoscope, display the first image data on the display of the video laryngoscope, receive a steering input on the display screen, and transmit a corresponding steering command to the endoscope via the first electrical interface.

11. The medical video system of claim 10, wherein the processor is configured to receive second image data from a camera of the video laryngoscope and display the second image data concurrently with displaying the first image data.

12. The medical video system of claim 1, wherein the second magnetic element is a permanent magnet or a magnetic material.

13. A medical device system comprising:
an endoscope connector comprising:
a port configured to at least partially enclose a proximal end of a steerable endoscope, the port comprising a first electrical interface inside the port that is electrically coupled with a second electrical interface of the endoscope connector that is outside the port;
a body attached along at least a portion of the length of the port, the body comprising:
a flange opposite a first edge of the body, and
a protrusion to be received by a recess of a video laryngoscope; and
a magnetic element attached to the body; and
the steerable endoscope, wherein the steerable endoscope comprises:
the proximal end, wherein the proximal end comprises a third electrical interface configured to be in contact with the first electrical interface when the proximal end is fully inserted into the port of the endoscope connector.

14. The medical device system of claim 13, wherein the body includes a cupped portion having an opening aligned with the second electrical interface.

15. The medical device system of claim 13, wherein the proximal end comprises a first rigid portion extending from a tip of the proximal end, a second rigid portion, and a flexible portion between the first rigid portion and the second rigid portion.

16. The medical device system of claim 15, wherein the second rigid portion comprises the third electrical interface and a motor configured to steer a distal end of the endoscope via a pull wire connected to the motor and the distal end.

17. The medical device system of claim 15, wherein the first rigid portion comprises a second motor configured to steer the distal end of the endoscope via a second pull wire connected to the second motor and the distal end.

18. The medical device system of claim 13, wherein the endoscope comprises a camera at the distal end.

19. A disposable connector for connecting an endoscope to a video laryngoscope, the disposable connector comprising:
a tubular housing defining a lumen and an opening to receive a proximal end of a steerable endoscope, the tubular housing comprising:
a first electrical interface exposed to the lumen;
a second electrical interface electrically coupled with the first electrical interface; and
a body attached to the tubular housing, the body comprising:
an upper planar portion;
a cupped portion protruding rearwardly from the upper planar portion and defining a partial cavity, wherein the second electrical interface is exposed to the cavity.

20. The disposable connector of claim 19, further comprising:
a flange opposite a first edge of the body, and
a protrusion to be received by a recess of a video laryngoscope; and
a magnetic element attached to the body.
